(19) United States
(12) Patent Application Publication
(10) Pub. No.: US 2025/0127432 A1
(43) Pub. Date: Apr. 24, 2025

(54) GLUCOSE MONITORING SYSTEM

(71) Applicant: Cirrus Logic International Semiconductor Ltd., Edinburgh (GB)

(72) Inventor: John P. LESSO, Edinburgh (GB)

(73) Assignee: Cirrus Logic International Semiconductor Ltd., Edinburgh (GB)

(21) Appl. No.: 18/912,735

(22) Filed: Oct. 11, 2024

Related U.S. Application Data
(60) Provisional application No. 63/592,357, filed on Oct. 23, 2023.

(51) Int. Cl. A61B 5/145; A61B 5/00; A61B 5/11

(52) U.S. Cl. CPC A61B 5/14532; A61B 5/11; A61B 5/4839; A61B 5/7264; A61B 5/7275; A61B 5/7405; A61B 5/742; A61B 5/7455; A61B 5/7465

(57) ABSTRACT

A glucose monitoring system comprising: a glucose sensor configured to output a glucose sensor output signal indicative of an estimate of a glucose level of a user of the glucose monitoring system; a movement sensor configured to output a movement sensor output signal indicative of detected movement of the user; and a controller configured to generate an estimate of a current glucose level of the user based on the glucose sensor output signal and the movement sensor output signal.

### FIELD OF THE INVENTION

The present disclosure relates to a glucose monitoring system.

### BACKGROUND

Wearable sensors are used for monitoring the health of the wearer. For example, wearable glucose sensors can be used to monitor a blood glucose level of the wearer.

Blood glucose levels can change rapidly in response to various stimuli and physiological conditions. The most common factor causing a swift alteration in glucose concentration is food intake, especially foods high in simple carbohydrates that are quickly absorbed into the bloodstream. Postprandially (i.e. after eating) blood glucose levels can surge within minutes and reach a peak typically within 30 minutes to 2 hours. Conversely, intense physical activity can lead to a rapid decrease in blood glucose as muscles consume glucose for energy.

Additionally, the release or administration of insulin, the hormone responsible for allowing cells to take in glucose from the blood, can lead to swift declines in blood glucose levels. These swift fluctuations underline the importance of frequent glucose monitoring, especially in individuals with diabetes, to avoid potentially dangerously high blood glucose levels (hyperglycemia) or low blood glucose levels (hypoglycemia).

Continuous glucose monitors (CGM) provide real-time tracking of blood glucose levels. However, there can be a time lag between the actual glucose level in venous blood and a corresponding glucose level reading output by a CGM, due to two effects.

The first is due to the fact the CGM device measures the glucose in interstitial fluid (ISF), rather than in venous blood. Interstitial fluid (ISF) and venous blood glucose measurements often exhibit a time discrepancy due to the physiological lag inherent in glucose transport dynamics. ISF glucose levels are derived from the fluid that surrounds the body's cells, whereas venous blood glucose levels directly measure glucose circulating in the bloodstream. The time lag arises because glucose must traverse capillary walls and equilibrate between the blood and interstitial spaces. The level of glucose in the blood can change rapidly, for example increasing after a meal or decreasing during intense exercise. However, it takes additional time for the change in blood glucose level change to be reflected in the ISF, leading to the observed lag between the actual blood glucose level and a corresponding reading from a CGM device. This delay varies between individuals and can be influenced by several factors, including blood flow rates, tissue properties, and metabolic activities. As there is not an exact correlation between the level of glucose in ISF and the level of glucose in venous blood, the glucose level reading output by a CGM device at a given point in time may not reflect the actual glucose level in venous blood at that point in time. As a result, when continuous glucose monitors (CGMs) that rely on ISF measurements are used, users are often advised to confirm critical decisions, like insulin dosing, with traditional fingerstick measurements that more closely represent blood glucose levels.

Secondly, there may be biochemical reactions within a sensor of the CGM device. For example, as the sensor is in the body there can be fibrinogen growth on the sensor which can lead to a time lag between the occurrence of a particular level of blood glucose and a corresponding measurement being output by the sensor.

The delay between a given level of glucose being present in venous blood and the output by a CGM device of a blood glucose reading that accurately reflects that blood glucose level can be of the order of 12-15 minutes. In the event that the blood glucose level is excessively high, the wearer may need to take action (e.g. move or exercise) to allow glucose to be used by muscle cells, thereby reducing blood glucose to a safe level. A delay in reporting the blood glucose level may be problematic, as it may delay the wearer from taking action to reduce their blood glucose level.

### SUMMARY

According to a first aspect, the invention provides a glucose monitoring system comprising: a glucose sensor configured to output a glucose sensor output signal indicative of an estimate of a glucose level of a user of the glucose monitoring system; a movement sensor configured to output a movement sensor output signal indicative of detected movement of the user; and a controller configured to generate an estimate of a current glucose level of the user based on the glucose sensor output signal and the movement sensor output signal.

The controller may be configured to control activation of the movement sensor and/or to control reading of data from the movement sensor based on the glucose sensor output signal.

The glucose monitoring system may be configured to activate the movement sensor if: a glucose level of the user, as determined based on the glucose sensor output signal, changes by more than a threshold amount; or a glucose level of the user, as determined based on the glucose sensor output signal, is either above a first threshold or below a second threshold, wherein the second threshold is lower than the first threshold.

The controller may be configured to read data from the movement sensor glucose if: a glucose level of the user, as determined based on the glucose sensor output signal, changes by more than a threshold amount; or a glucose level of the user, as determined based on the glucose sensor output signal, is either above a first threshold or below a second threshold, wherein the second threshold is lower than the first threshold.

The glucose monitoring system may further comprise an output transducer subsystem configured to provide an output for the user based on the glucose sensor output signal and the movement sensor output signal.

The output transducer subsystem may comprise an audio transducer and/or a haptic transducer.

The glucose monitoring system may further comprise a communications subsystem configured to communicate with an external device to transmit data and/or alerts to the external device.

The movement sensor may comprise an accelerometer.

The accelerometer may be configured to generate an analog output signal. An output of the accelerometer may be coupled to an analog to digital converter (ADC).

The ADC may be configured to output an ADC output signal on detection of a significant change in the analog output signal of the accelerometer.

The controller may be configured to trigger an output signal based on one or more of: the glucose sensor output signal; the movement output signal; and a combination or fusion of the glucose sensor output signal and the movement sensor output signal.

The output may comprise one or more of: an audio output signal for an audio output transducer of the glucose monitoring system; a haptic output signal for a haptic output transducer of the glucose monitoring system; and a control signal for transmission to an external device with which the glucose monitoring system communicates via a communications subsystem of the glucose monitoring system, the control signal being configured to trigger an audible and/or visible and/or haptic output of the external device.

The glucose monitoring system may further comprise a predictor configured to generate a prediction of a current and/or future glucose level of the user based on the glucose sensor output signal and the movement sensor output signal.

The predictor may comprise a trained neural network, artificial intelligence or machine learning processor trained to generate the prediction of the current and/or future glucose level of the wearer based on the glucose sensor output signal and the movement sensor output signal.

The glucose monitoring system may be configured to output a signal to an external insulin pump to control an amount of insulin delivered to the user by the external insulin pump based on the glucose sensor output signal and the movement sensor output signal.

The glucose monitoring system may be a wearable glucose monitoring system.

According to a second aspect, the invention provides a glucose monitoring system comprising: a glucose sensor configured to output a glucose sensor output signal indicative of an estimate of a glucose level of a wearer of the glucose monitoring system; an accelerometer configured to output an accelerometer output signal indicative of movement of a wearer of the glucose monitoring system; and a controller operable to: monitor a wearer glucose level estimate generated based on the glucose sensor output signal; monitor a wearer movement estimate generated based on the accelerometer output signal; determine a wearer health marker based on the monitored wearer movement estimate; and combine the monitored wearer glucose level estimate with the wearer health marker to generate a prediction of a current and/or future glucose level of the wearer.

The glucose monitoring system may comprise a predictor configured to predict the current and/or future glucose level of the wearer based on the monitored wearer glucose level estimate and the wearer health marker.

The predictor may comprise a trained neural network, artificial intelligence or machine learning processor trained to generate the prediction of the current and/or future glucose level of the wearer based on the monitored wearer glucose level estimate and the wearer health marker.

According to a third aspect, the invention provides an integrated circuit for a glucose monitoring system, the integrated circuit comprising: a movement sensor; and a controller, wherein the controller is configured to: receive a movement output signal indicative of detected movement of a user of the glucose monitoring system; receive, from a glucose sensor of the glucose monitoring system, a glucose sensor output signal indicative of an estimate of a glucose level of the user; and generate an estimate of a current and/or future glucose level of the user based on the glucose sensor output signal and the movement sensor output signal.

According to a fourth aspect, the invention provides a glucose monitoring system comprising: a glucose sensor configured to output a glucose sensor output signal indicative of an estimate of a glucose level of a user of the glucose monitoring system; and a movement sensor configured to output a movement sensor output signal indicative of detected movement of the user, wherein the system is configured to produce feedback for the user based on the glucose sensor output signal and the movement sensor output signal.

According to a fifth aspect, the invention provides a glucose monitoring system comprising: a glucose sensor configured to output a glucose sensor output signal indicative of an estimate of a glucose level of a user of the glucose monitoring system; a movement sensor configured to output a movement sensor output signal indicative of detected movement of the user; and a controller configured to configured to control activation of the movement sensor and/or to control reading of data from the movement sensor based on the glucose sensor output signal.

According to a sixth aspect, the invention provides an integrated circuit for a glucose monitoring system, the integrated circuit comprising: a movement sensor; and a controller, wherein the controller is configured to control activation of the movement sensor and/or to control reading of data from the movement sensor based a glucose sensor output signal received from a glucose sensor of the glucose monitoring system.

According to a seventh aspect, the invention provides a glucose monitoring system comprising: a glucose sensor configured to output a glucose sensor output signal indicative of an estimate of a glucose level of a user of the glucose monitoring system; an accelerometer configured to output an accelerometer output signal indicative of detected movement of the user; and

a predictor configured to generate a prediction of a current and/or future glucose level of the user based on the glucose sensor output signal and the movement sensor output signal.

According to an eighth aspect, the invention provides an integrated circuit for a glucose monitoring system, the integrated circuit comprising: an accelerometer configured to output an accelerometer output signal indicative of detected movement of the user; and a predictor, wherein the predictor is configured to generate a prediction of a current and/or future glucose level of user of the glucose monitoring system based on the accelerometer output signal and a glucose sensor output signal received from a glucose sensor of the glucose monitoring system.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the invention will now be described, strictly by way of example only, with reference to the accompanying drawings, of which:

FIG. 1 is a schematic representation of a glucose monitoring system according to the present disclosure;

FIG. 2 is a schematic representation of an alternative glucose monitoring system according to the present disclosure;

FIG. 3 is a schematic representation of a further example glucose monitoring system;

FIG. 4 is a schematic representation of a further example glucose monitoring system according to the present disclosure; and

FIG. 5 is a schematic diagram showing a glucose monitoring system according to the present disclosure communicating with an insulin pump.

### DETAILED DESCRIPTION

Patients with unstable glucose levels could benefit from real-time recommendations or accurate control of insulin dosage based on their current activity level.

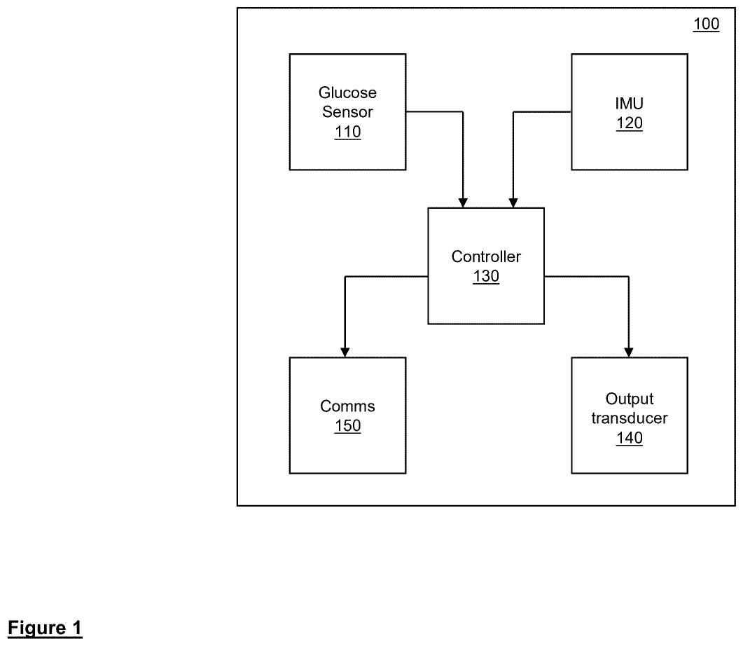

FIG. 1 is a schematic representation of an example wearable glucose monitoring system. The system, shown generally at 100 in FIG. 1, includes a glucose sensor 110, a movement sensor 120, a controller 130, and an output transducer subsystem 140. The system 100 may further include a communications subsystem 150.

The glucose sensor 110 is configured to provide an estimate or indication of a blood glucose level of a user (wearer) of the system 100. The glucose sensor 110 may be implemented using any suitable glucose sensing technology, e.g. a potentiostat sensor coupled to an electrochemical sensor configured to be placed, in use, on the user's body to measure the level of glucose in the interstitial fluid of the user. The glucose sensor 110 is configured to output a glucose sensor output signal indicative of the estimate of the blood glucose level of the user to the controller 130.

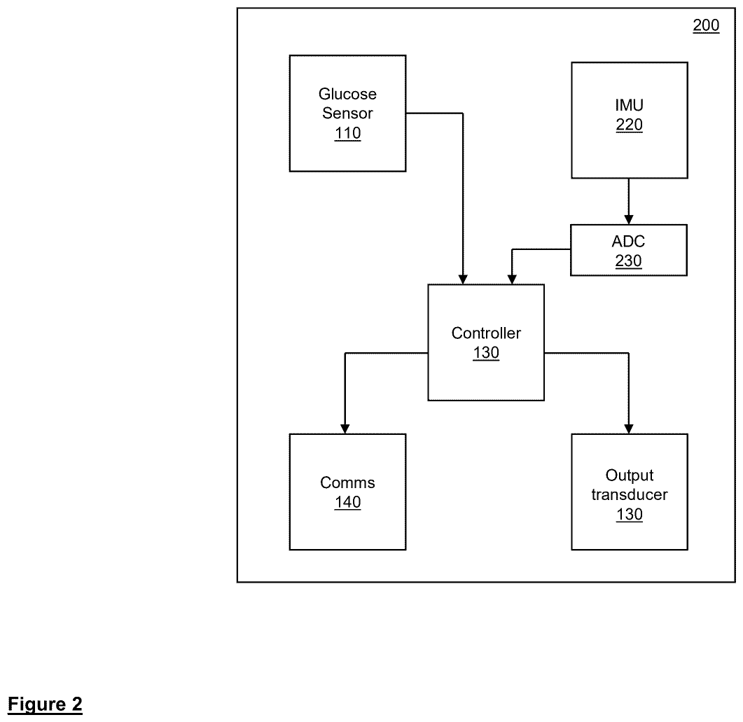

The movement sensor 120 is configured to provide an estimate of movement of the user (wearer). The movement sensor 120 may comprise, for example, an accelerometer (e.g. a three-axis accelerometer) or inertial measurement unit (IMU). The movement sensor 120 is configured to output a movement sensor output signal indicative of movement of the user to the controller 130. In some examples the movement sensor 120 provides a digital output signal to the controller 130. In other examples (described in more detail below with reference to FIG. 2), the movement sensor 120 may comprise an analog movement sensor configured to output an analog output signal.

The controller 130 is configured to monitor the glucose sensor output signal output by the glucose sensor 110 and the movement sensor output signal output by the movement sensor 120. The controller 130 is configured to evaluate glucose data based on the glucose sensor output signal. The controller 130 may be further configured to activate the movement sensor 120 or to read data from the movement sensor 120 (e.g. by sampling the movement sensor output signal) based on the glucose data, and produce feedback for the user based on combined glucose and movement data.

The output transducer subsystem 140 may comprise one or more output transducers, for example an audio transducer such as a loudspeaker and/or a haptic transducer such as a vibrational actuator. The output transducer subsystem 140 is operable to generate an output for a wearer of the system 100 in response to a control signal output by the controller 130 if it is determined that a monitored glucose and/or movement level at a level requiring attention from the wearer, e.g. dangerously high or low levels of glucose.

The communications subsystem 150 is configured to communicate, via a wired or wireless connection, with a separate device, external to the system 100, to communicate data and/or alerts from the system 100 to the external device for notification and/or display to the user of the system 100. The external device may be a host device such as a mobile telephone, laptop or tablet computer. The communications subsystem 150 may be configured to communicate with the external device using a wireless connection such as Bluetooth®, Wi-Fi, near field communications (NFC), radio frequency identification (RFID) or the like. Additionally or alternatively, the communications subsystem 150 may be configured to communicate with the external device using a wired connection such as universal serial bus (USB).

The controller 130 may be configured to adaptively adjust operation of the movement sensor 120 (i.e. activation and/or sampling of the movement sensor 120) based on the monitored output of the glucose sensor 110.

In one example, the controller 130 may be configured to activate the movement sensor 120 when an inferred blood glucose level (as may be determined, for example, by the controller 130 based on the glucose sensor output signal output by the glucose sensor 110) changes by more than an allowable amount over a set time period. For example, the controller 130 may be configured to determine a difference between a glucose level at a first point in time and at a second point in time, and to activate the movement sensor if the difference exceeds a threshold.

In another example, the controller 130 may be configured to activate the movement sensor 120 when the inferred blood glucose level is either above a first threshold or below a second threshold which is lower than the first threshold.

In a further example, the controller 130 may be configured to fuse or combine movement data (as may be determined, for example, by the controller 130 based on the movement sensor output signal output by the movement sensor 120), and the inferred glucose level (as may be determined, for example, by the controller 130 based on the glucose sensor output signal output by the glucose sensor 110) to generate an (improved) estimate of a current blood glucose level of the user, thereby reducing the time lag between the occurrence of a particular level of blood glucose and the output of a corresponding blood glucose measurement inherent in known CGM systems.

The system 100 (e.g. the controller 130) can learn over time how quickly a user's glucose levels change with a given intensity of movement as indicated by the signal output by the movement sensor 120. For example, when the user has a high glucose level and starts moving, initially there is a drop in blood glucose level that may not immediately be detected by the glucose sensor 120, but correlates with a movement intensity vector that can be derived, e.g. by the controller 130, from the movement sensor output signal output by the movement sensor 120. By looking at the glucose level at a given lag the system 100 (e.g. the controller 130) can learn the correlation between the movement intensity level and the drop in blood glucose level, and, for a given intensity of movement, can predict a blood glucose level more accurately.

To this end, the system 100 may comprise a predictor for predicting a user's current and/or future blood glucose level (e.g. the user's current blood glucose level or the user's blood glucose level at a given point in the future, e.g. one minute, half an hour, or one hour in the future) based on a current monitored blood glucose level (as indicated by the glucose sensor output signal) and a detected level of movement (as indicated by the movement sensor output signal).

The predictor may be a standalone element of the system 100, or alternatively may be provided as part of the controller 130. The predictor may comprise, for example, a trained neural network, artificial intelligence (AI) or other machine learning processor trained using suitable training data (e.g. glucose data and movement data gathered over time for the user of the system 100) to predict the effect of movement or other physical activity on the user's blood glucose level and thus generate a prediction of the user's future blood glucose level. This prediction may be transmitted to the external device via the communications subsystem 150. Additionally or alternatively, an output indicative of the predicted future blood glucose level (e.g. an audible output or a haptic output) may be provided to the user via the output transducer subsystem 140.

An example of an alert which can be generated using the above-described system is an alert or prompt to encourage the user to engage in physical activity, if the glucose sensor output signal output by the glucose sensor 110 is indicative of a high blood glucose level and the movement sensor output signal output by the movement sensor 120 is indicative of little or no movement by the user.

A further example of an alert which can be generated using the above-described system is an alert or prompt to encourage the user to sit down or rest, if the output signal the glucose sensor 110 is indicative of a low blood glucose level and the output signal of the movement sensor 120 is indicative of movement by the user.

In a further example, an alert could be generated based on movement data or glucose data alone. For example, if the if the glucose sensor output signal output by the glucose sensor 110 is indicative of a high blood glucose level, the system 100 may generate and output an alert or prompt to encourage the user to engage in physical activity. Similarly, if movement sensor output signal output by the movement sensor 120 is indicative of a period of intensive movement by the user, the system 100 may generate and output an alert or prompt to encourage the user to sit down or rest.

Such alerts may be audible and/or haptic alerts output by the output transducer subsystem 140. Additionally or alternatively, the communications subsystem 150 may transmit a control signal to the external device to cause the external device to output an alert or prompt, which may be, for example, one or more of a visible alert or prompt such as a message displayed on a screen of the external device, an audible alert or prompt output by a speaker of the external device, and a haptic alert or prompt output by a haptic transducer of the external device.

In some implementations, the movement sensor 120 may be an activity-triggered movement sensor, e.g. an activity-triggered accelerometer. In such implementations the movement sensor 120 may be held in low-power or inactive mode and only activated in response to a trigger signal, e.g. as may be output by the controller 130 when a rapid change in inferred blood glucose levels is detected over a set period.

The system 100 (e.g. the controller 130) may fuse data derived from or based on the movement sensor output signal output by the movement sensor 120 with glucose readings derived from or based on the glucose sensor output signal output by the glucose sensor 110. Movement data can, for instance, be used (e.g. by the controller 130) to predict an impending drop in glucose levels, and in such circumstances the system 100 may providing an early warning to the user, e.g. via an alert of the kind described above. Over time, the system 100 (e.g. the controller 130) can learn the relationship between movement patterns and glucose variations, refining its predictions and feedback.

FIG. 2 is a schematic representation of an alternative example wearable glucose monitoring system. The alternative system, shown generally at 200 in FIG. 2, includes many features in common with the system 100 of FIG. 1. Such common features are denoted by common reference numerals in FIGS. 1 and 2, and will not be described in detail here, for the sake of clarity and brevity.

The system 200 of FIG. 2 differs from the system 100 of FIG. 1 in that, instead of a digital movement sensor 120, the system 200 includes a movement sensor 220 having an analog output that is coupled to an input of an analog to digital converter (ADC) 230. An output of the ADC 230 is coupled to an input of the controller 130 so as to supply a digital signal indicative of movement of the user to the controller 130.

The movement sensor 220 may comprise, for example, an accelerometer (e.g. a three-axis accelerometer) or inertial measurement unit (IMU).

The ADC 230 may comprise an event-driven ADC configured to detect relatively significant changes in the input analog signal received from the movement sensor 220 (e.g. a change in a characteristic or property of the input analog signal such as a magnitude of the input analog signal that exceeds a threshold, or that exceeds a threshold for a predetermined period of time), and to generate a digital ADC output signal when significant changes are detected in the input analog signal, which may be indicative, for example, that significant movement of the device or wearer of the device has been detected.

The movement sensor 220 can provide continuous monitoring of user movement. If the ADC 230 is an event-driven ADC 230, it can detect significant changes in user movement, and can also detect long periods of inactivity of the user (wearer) of the device 200. Based on the this and glucose readings derived from or based on the glucose sensor output signal output by the glucose sensor 110, the system 200 can provide alerts or prompts of the kind described above with reference to FIG. 1. Thus, an alert may be provided (as an audible or haptic output of the output transducer subsystem 140 and/or as an output of the external device which the system 200 communicates via the communications subsystem 150) when glucose is high and activity drops, and another alert may be provided (again, as an audible or haptic output of the output transducer subsystem 140 and/or as an output of the external device which the system 200 communicates via the communications subsystem 150) when glucose is low and activity increases.

Quantifying movement using a 3-axis accelerometer worn on the body involves measuring accelerations along three orthogonal axes, typically denoted as X, Y, and Z. These axes correspond to the three spatial dimensions: side-to-side (lateral), up-and-down (vertical), and forward-and-backward (longitudinal). Once the accelerometer is securely positioned on the body, it continuously measures the gravitational and dynamic accelerations experienced along each axis as the individual moves. By integrating these measurements over time, one can obtain the velocity and ultimately the displacement in each direction.

To effectively quantify movement, the raw data is often processed to filter out noise, and various algorithms or mathematical models are employed to derive metrics such as step count, posture, or activity intensity. Furthermore, the magnitude of the overall acceleration vector can be calculated using the Pythagorean theorem as the square root of the sum of the squares of the individual axes accelerations. This resultant magnitude provides an aggregate measure of movement intensity, irrespective of the direction, and is especially valuable for activity recognition and quantifying overall physical activity levels.

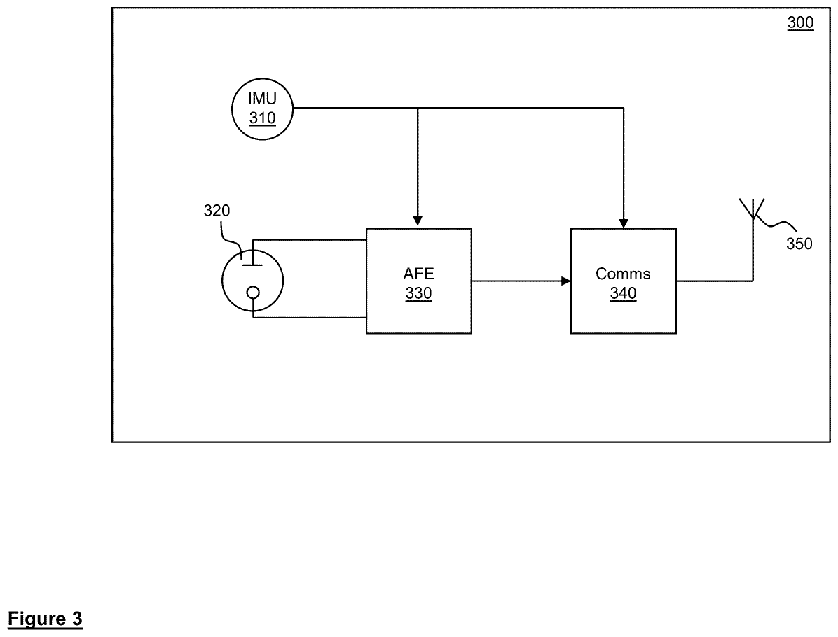

FIG. 3 is a schematic representation of a further example wearable glucose monitoring system. The system, shown generally at 300 in FIG. 3, includes a movement sensor 310, which may be an accelerometer or IMU, and a glucose sensor, shown here as an electrochemical cell CGM 310 coupled with an analog front end (AFE) 330. The system further includes a communications subsystem 340, shown here as a wireless communications subsystem (e.g. based on Bluetooth®, Wi-Fi, near field communications (NFC), radio frequency identification (RFID) or the like) coupled to an antenna 350.

The movement sensor 310 in this example has a digital output and is coupled to one or both of the AFE 330 and the communications subsystem 340 such that it can output a digital output signal to either of both of the AFE 330 and the communications subsystem 340.

In this example, the movement sensor 310 can be used to detect and/or monitor low frequency sounds (e.g. sub 500 Hz) in the body via a mixture of soft body and bone conduction. The mechano-acoustic propagation of sounds from the cardiopulmonary systems can be used to give insight into heart and lung function. Additionally, if the system 300 is mounted on the user's abdomen, gut sounds may be detected and/or monitored. Alerts or prompts may be generated based on such detected sounds.

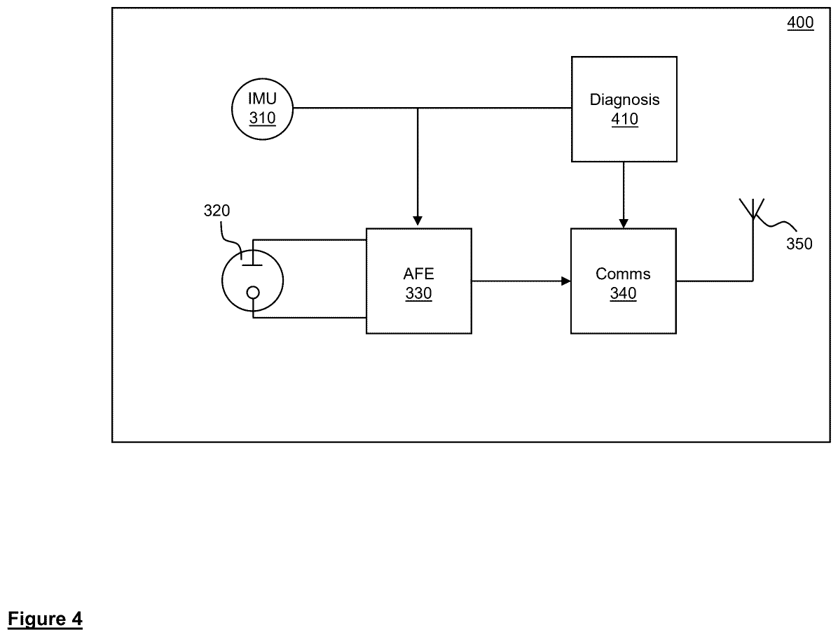

FIG. 4 is a schematic representation of a further example wearable glucose monitoring system that can generate alerts or prompts based on detected body sounds, in combination with data relating to a blood glucose level.

The system, shown generally at 400 in FIG. 4, has a number of features in common with the system 300 of FIG. 3. Such common features are denoted by common reference numerals in FIGS. 3 and 4, and will not be described again here for the sake of clarity and brevity.

The system 400 differs from the system 300 in that it further includes a diagnosis block 410. The diagnosis block comprises a processor or controller, such as a microprocessor, microcontroller, digital signal processor or the like. The diagnosis block 410 is coupled to the communications subsystem 340 such that it can transmit data to an external device such as a mobile telephone, tablet or laptop computer or the like via the communications subsystem 340.

The diagnosis block 410 is coupled to the movement sensor 310 so as to receive an output signal from the movement sensor 310. Based on the received movement sensor output signal, the diagnosis block 410 is able to monitor cardiopulmonary and/or gut conditions.

In examples in which the diagnosis block 410 comprises a digital signal processor (DSP), the movement sensor 310 can be provided as a fully-analog component providing an analog output, and the diagnosis block 410 can have an ADC to convert the analog output signal output by the movement sensor 310 into a digital signal, e.g. to save power and/or cost.

Alternatively, the diagnosis block 410 may be implemented as an analog computing block arranged to receive an analog signal from the movement sensor 310 directly.

It will be understood that the diagnosis block 410 may be implemented as a stand-alone processor, or may be implemented as part of a central controller processor of the system 400, which may also receive the output from the AFE 330.

Depending on where the system 400 is worn or mounted on the user's body, the data relating to the several physiological variables may be tracked or monitored. For example, a user's heartbeat may be determined and monitored by measuring a phonocardiogram (PCG) that may be derived by the diagnosis block 410 from cardiac sounds detected by the movement sensor 310. Respiration rate can be tracked or monitored based on respiratory sounds detected by the movement sensor 310. Respiratory disease can be tracked and identified, again based on respiratory sounds detected by the movement sensor 310. Gut sounds detected by the movement sensor 310 can be used to identify when the user is eating, and can also be used to identify defecation.

These data can be combined with glucose level data or CGM measurements derived from or based on the glucose sensor output signal output by the glucose sensor to improve outcomes.

For example, gut sounds can be used by the system 400 to detect eating, which can be used to predict a rise in the user's blood glucose level. In response to a predicted increase in the blood glucose level, the system 400 may generate an alert or prompt (of the kind described above with reference to FIG. 1) to prompt the user to undertake physical activity.

Other suitable alerts may be generated based on the other physiological variables that are tracked or monitored by the diagnosis block 410. For example, an increased respiration rate may be used to infer that the user is undertaking physical activity which may lead to a drop in blood glucose level. In response to a predicted drop in blood glucose level, the system 400 may generate an alert or prompt (of the kind described above with reference to FIG. 1) to prompt the user to rest and/or eat.

Some diabetes patients are provided with automatic insulin delivery (AID) systems, which deliver measured doses of insulin to the patient in response to changes in the patient's blood glucose level. Such system typically comprise a continuous glucose monitor and an insulin pump. The continuous glucose monitor detects the patient's blood glucose level (typically indirectly via detection of the level of interstitial glucose) and communicates with the insulin pump to adjust the amount of insulin delivered to the patient according to the detected blood glucose level.

The wearable glucose monitoring system of the present disclosure may be configured to communicate with the insulin pump of an AID system to control more accurately the amount of insulin delivered to the patient, by taking into account not only a detected blood glucose level, but also movement of the patient.

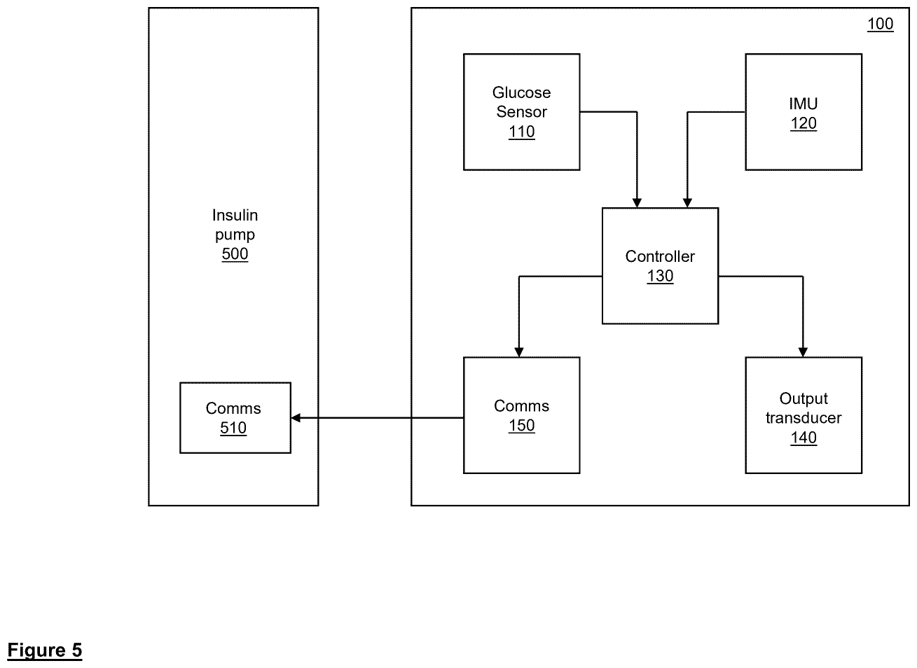

FIG. 5 is a schematic diagram showing the wearable glucose monitoring system 100 of FIG. 1 communicating with an insulin pump 500 via a wired or wireless communications link between the communications subsystem 150 of the system 100 and a communications subsystem 510 of the insulin pump 500.

As described above with reference to FIG. 1, the controller 130 is configured receive a signal indicative of a user's blood glucose level from the glucose sensor 110 and a signal indicative of movement of the user from the movement sensor 120. The controller 130 can combine glucose measurement data derived from or based on the signal received from the glucose sensor 110 with movement data derived from or based on the signal received from the movement sensor 120, and may use the resulting combination of data to output (via the communications subsystem 150) a control signal to control the amount of insulin delivered by the insulin pump.

For example, if the signal output by the glucose sensor 110 is indicative of a high blood glucose level and the signal output by the movement sensor 120 is indicative of little or no movement, the controller 130 may generate and output a control signal to the insulin pump 500 (via the communications subsystem 150) to cause the insulin pump to deliver an increased amount of insulin to the user. In contrast, if the signal output by the glucose sensor 110 is indicative of a low blood glucose level and the signal output by the movement sensor 120 is indicative of movement by the user, the controller 130 may generate and output a control signal to the insulin pump 500 (via the communications subsystem 150) to cause the insulin pump to deliver a reduced amount of insulin to the user, or to deliver no insulin to the user.

Similarly, the alternative system 400 shown in FIG. 4 may communicate with an insulin pump 500 to control the amount of insulin delivered to a user based on a combination of blood glucose data derived from or based on an output of the glucose sensor (made up of the continuous glucose monitor 320 and AFE 330) and physiological sound data derived from or based on the signal output by the movement sensor 310.

The systems described above with reference to FIGS. 1-5 may be implemented, at least in part, in integrated circuits.

For example, the movement sensor 120, controller 130 and communications subsystem of the system 100 of FIGS. 1 and 5 may be implemented in a single integrated circuit (IC), while the glucose sensor 110 and output transducer subsystem 140 may be external to the IC and coupled to suitable coupling terminals (e.g. balls, pads or pins) of the IC.

Similarly, the movement sensor 120, controller 130, ADC 220 and communications subsystem of the system 200 of FIG. 2 may be implemented in a single integrated circuit (IC), while the glucose sensor 110 and output transducer subsystem 140 may be external to the IC and coupled to suitable coupling terminals (e.g. balls, pads or pins) of the IC.

Likewise, the movement sensor 310, AFE 330 and communications subsystem 340 of the system 300 of FIG. 3 may be implemented in a single IC, with the continuous glucose monitor 320 and antenna 350 being external to the IC and coupled to suitable coupling terminals (e.g. balls, pads or pins) of the IC.

Similarly, the movement sensor 310, AFE 330 communications subsystem 340 and diagnosis block 410 of the system 400 of FIG. 4 may be implemented in a single IC, with the continuous glucose monitor 320 and antenna 350 being external to the IC and coupled to suitable coupling terminals (e.g. balls, pads or pins) of the IC.

As will be appreciated from the foregoing disclosure, the systems disclosed herein permit improved regulation of blood glucose, as the combination of blood glucose data with movement data or physiological sound data enables a more accurate prediction or estimate of the user's current blood glucose level than is possible using known continuous glucose monitoring systems. By prompting the user to take appropriate action or controlling an insulin in response to this combination of data, the user's blood glucose level can be controlled more accurately and in a more timely manner than is possible using existing continuous glucose monitoring systems.

The skilled person will recognise that some aspects of the above-described apparatus and methods may be embodied as processor control code, for example on a non-volatile carrier medium such as a disk, CD- or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit) or FPGA (Field Programmable Gate Array). Thus the code may comprise conventional program code or microcode or, for example code for setting up or controlling an ASIC or FPGA. The code may also comprise code for dynamically configuring re-configurable apparatus such as re-programmable logic gate arrays. Similarly the code may comprise code for a hardware description language such as Verilog™ or VHDL (Very high speed integrated circuit Hardware Description Language). As the skilled person will appreciate, the code may be distributed between a plurality of coupled components in communication with one another. Where appropriate, the embodiments may also be implemented using code running on a field-(re)programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general purpose processor or the like. A module may itself comprise other modules or functional units. A module may be provided by multiple components or sub-modules which need not be co-located and could be provided on different integrated circuits and/or running on different processors.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art, and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

1. A glucose monitoring system comprising:
a glucose sensor configured to output a glucose sensor output signal indicative of an estimate of a glucose level of a user of the glucose monitoring system;
a movement sensor configured to output a movement sensor output signal indicative of detected movement of the user; and
a controller configured to generate an estimate of a current glucose level of the user based on the glucose sensor output signal and the movement sensor output signal.

2. The glucose monitoring system of claim 1, wherein the controller is configured to control activation of the movement sensor and/or to control reading of data from the movement sensor based on the glucose sensor output signal.

3. The glucose monitoring system of claim 2, wherein the glucose monitoring system is configured to activate the movement sensor if:
a glucose level of the user, as determined based on the glucose sensor output signal, changes by more than a threshold amount; or
a glucose level of the user, as determined based on the glucose sensor output signal, is either above a first threshold or below a second threshold, wherein the second threshold is lower than the first threshold.

4. The glucose monitoring system of claim 2, wherein the controller is configured to read data from the movement sensor glucose if:
a glucose level of the user, as determined based on the glucose sensor output signal, changes by more than a threshold amount; or
a glucose level of the user, as determined based on the glucose sensor output signal, is either above a first threshold or below a second threshold, wherein the second threshold is lower than the first threshold.

5. The glucose monitoring system of claim 1, further comprising an output transducer subsystem configured to provide an output for the user based on the glucose sensor output signal and the movement sensor output signal.

6. The glucose monitoring system of claim 5, wherein the output transducer subsystem comprises an audio transducer and/or a haptic transducer.

7. The glucose monitoring system of claim 1, further comprising a communications subsystem configured to communicate with an external device to transmit data and/or alerts to the external device.

8. The glucose monitoring system of claim 1, wherein the movement sensor comprises an accelerometer.

9. The glucose monitoring system of claim 8, wherein the accelerometer is configured to generate an analog output signal, and wherein an output of the accelerometer is coupled to an analog to digital converter (ADC).

10. The glucose monitoring system of claim 9, wherein the ADC is configured to output an ADC output signal on detection of a significant change in the analog output signal of the accelerometer.

11. The glucose monitoring system of claim 1, wherein the controller is configured to trigger an output signal based on one or more of:
the glucose sensor output signal;
the movement output signal; and
a combination or fusion of the glucose sensor output signal and the movement sensor output signal.

12. The glucose monitoring system of claim 11, wherein the output comprises one or more of:
an audio output signal for an audio output transducer of the glucose monitoring system;
a haptic output signal for a haptic output transducer of the glucose monitoring system; and
a control signal for transmission to an external device with which the glucose monitoring system communicates via a communications subsystem of the glucose monitoring system, the control signal being configured to trigger an audible and/or visible and/or haptic output of the external device.

13. The glucose monitoring system of claim 1, further comprising a predictor configured to generate a prediction of a current and/or future glucose level of the user based on the glucose sensor output signal and the movement sensor output signal.

14. The glucose monitoring system of claim 13, wherein the predictor comprises a trained neural network, artificial intelligence or machine learning processor trained to generate the prediction of the current and/or future glucose level of the wearer based on the glucose sensor output signal and the movement sensor output signal.

15. The glucose monitoring system of claim 1, wherein the glucose monitoring system is configured to output a signal to an external insulin pump to control an amount of insulin delivered to the user by the external insulin pump based on the glucose sensor output signal and the movement sensor output signal.

16. The glucose monitoring system of claim 1, wherein the glucose monitoring system is a wearable glucose monitoring system.

17. A glucose monitoring system comprising:
a glucose sensor configured to output a glucose sensor output signal indicative of an estimate of a glucose level of a wearer of the glucose monitoring system;
an accelerometer configured to output an accelerometer output signal indicative of movement of a wearer of the glucose monitoring system; and
a controller operable to:
monitor a wearer glucose level estimate generated based on the glucose sensor output signal;
monitor a wearer movement estimate generated based on the accelerometer output signal;
determine a wearer health marker based on the monitored wearer movement estimate; and
combine the monitored wearer glucose level estimate with the wearer health marker to generate a prediction of a current and/or future glucose level of the wearer.

18. The glucose monitoring system of claim 17, wherein the glucose monitoring system comprises a predictor configured to predict the current and/or future glucose level of the wearer based on the monitored wearer glucose level estimate and the wearer health marker.

19. The glucose monitoring system of claim 18, wherein the predictor comprises a trained neural network, artificial intelligence or machine learning processor trained to generate the prediction of the current and/or future glucose level of the wearer based on the monitored wearer glucose level estimate and the wearer health marker.

20. An integrated circuit for a glucose monitoring system, the integrated circuit comprising:
a movement sensor; and
a controller,
wherein the controller is configured to:
receive a movement output signal indicative of detected movement of a user of the glucose monitoring system;
receive, from a glucose sensor of the glucose monitoring system, a glucose sensor output signal indicative of an estimate of a glucose level of the user; and
generate an estimate of a current and/or future glucose level of the user based on the glucose sensor output signal and the movement sensor output signal.

21. A glucose monitoring system comprising:
a glucose sensor configured to output a glucose sensor output signal indicative of an estimate of a glucose level of a user of the glucose monitoring system; and
a movement sensor configured to output a movement sensor output signal indicative of detected movement of the user,
wherein the system is configured to produce feedback for the user based on the glucose sensor output signal and the movement sensor output signal.

22. A glucose monitoring system comprising:
a glucose sensor configured to output a glucose sensor output signal indicative of an estimate of a glucose level of a user of the glucose monitoring system;
a movement sensor configured to output a movement sensor output signal indicative of detected movement of the user; and
a controller configured to configured to control activation of the movement sensor and/or to control reading of data from the movement sensor based on the glucose sensor output signal.

23. An integrated circuit for a glucose monitoring system, the integrated circuit comprising:
a movement sensor; and
a controller,
wherein the controller is configured to control activation of the movement sensor and/or to control reading of data from the movement sensor based a glucose sensor output signal received from a glucose sensor of the glucose monitoring system.

24. A glucose monitoring system comprising:
a glucose sensor configured to output a glucose sensor output signal indicative of an estimate of a glucose level of a user of the glucose monitoring system;
an accelerometer configured to output an accelerometer output signal indicative of detected movement of the user; and
a predictor configured to generate a prediction of a current and/or future glucose level of the user based on the glucose sensor output signal and the movement sensor output signal.

25. An integrated circuit for a glucose monitoring system, the integrated circuit comprising:
an accelerometer configured to output an accelerometer output signal indicative of detected movement of the user; and
a predictor,
wherein the predictor is configured to generate a prediction of a current and/or future glucose level of user of the glucose monitoring system based on the accelerometer output signal and a glucose sensor output signal received from a glucose sensor of the glucose monitoring system.
